(19) United States
(12) Patent Application Publication
(10) Pub. No.: US 2006/0024707 A1
(43) Pub. Date: Feb. 2, 2006

(54) Luminescent polymers and methods of use thereof

(76) Inventors: Robert Deans, Grafton, MA (US); Lawrence F. Hancock, N. Andover, MA (US); Joong Ho Moon, Chestnut Hill, MA (US); Jerry R. Malayer, Stillwater, OK (US); Cyril R. Clarke, Perkins, OK (US); Jean M. Clarke, Perkins, OK (US); Akhilesh Ramachandran, Stillwater, OK (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 11/148,151

(22) Filed: Jun. 8, 2005

Related U.S. Application Data
(63) Continuation-in-part of application No. 10/325,660, filed on Dec. 19, 2002.
(63) Continuation-in-part of application No. PCT/US03/40566, filed on Dec. 19, 2003.
Application No. PCT/US03/40566 is a continuation-in-part of application No. 10/325,660, filed on Dec. 19, 2002.

(51) Int. Cl. C12Q 1/68; C08G 63/48; C08G 63/91

(52) U.S. Cl. 435/6; 525/54.2

(57) ABSTRACT

The present invention involves a series of articles, compositions, methods, and kits. Some aspects of the invention include articles such as particles, sols, blends, dispersions, films, or microarrays that comprise luminescent polymers, as well as methods for making and using such articles. In some cases, the luminescent polymer may be characterized in part by having a delocalized π-orbital structure, which can allow the polymer to have a high degree of luminosity. The polymers of this invention may also have, in some embodiments, bulky substituents to prevent intermolecular π-π interactions that can decrease luminosity. Some articles may include more than one luminescent polymer, for example, a first polymer that absorbs energy and directs the energy to a second polymer that releases the energy. The articles of the invention may be used, in certain embodiments, to detect the presence of other compounds such as single molecules, proteins, or specific nucleic acid sequences, as well as cells, bacteria, and viruses. In one set of embodiments, the luminescent polymers are associated with an entity that can interact with, for example, a nucleic acid or a protein. The association may be direct, or through an energy transfer pathway. The entity can be, for example, a nucleic acid, a charged surface, an intercalating agent, or an entity that releases a quenching agent that interacts with the luminescent polymer.

### RELATED APPLICATIONS

This application is a continuation-in-part of U.S. patent application Ser. No. 10/325,660, filed Dec. 19, 2002, entitled “Luminescent Polymers and Methods of Use Thereof,” by Deans, et al. This application is also a continuation-in-part of International Patent Application Serial No. PCT/US03/40566, filed Dec. 19, 2003, entitled “Luminescent Polymers and Methods of Use Thereof,” by Deans, et al., which application is a continuation-in-part of said U.S. patent application Ser. No. 10/325,660. Each of these applications is incorporated herein by reference.

### BACKGROUND

1. Field of the Invention

This invention generally relates to luminescent polymers, articles including luminescent polymers, and methods of making and using such articles, for example, for the detection of nucleic acids, proteins, small molecules, and the like.

b 2. Description of the Related Art

There is a high demand for chemical sensor devices for detecting low concentration levels of analytes present in the liquid and gaseous phase. Specificity to particular analytes is also generally desired. Certain chemical sensor devices may involve luminescent materials, as luminescence lifetimes and intensities may be sensitive to the presence of external species or analytes. Luminescent polyelectrolytes such as poly(phenylene)s or poly(phenylene vinylene)s have been described in, for example, Harrison, et al., J. Am. Chem. Soc., 122: 8561 (2000); Gaylord, et al., J. Am. Chem. Soc., 123: 6417 (2001). Various techniques can be used to detect and monitor luminescent polyelectrolytes, such as ordinary light or fluorescence microscopy, laser scanning confocal microscopy, or fluorescence spectroscopy and related techniques.

### SUMMARY OF THE INVENTION

This invention relates to articles including luminescent polymers, and methods of making and using such articles, for example, in the detection of nucleic acids, proteins, small molecules, and the like. The subject matter of this application involves, in some cases, interrelated products, alternative solutions to a particular problem, and/or a plurality of different uses of a single system or article.

In one aspect, the invention comprises an article. In one set of embodiments, the article includes a composition comprising a luminescent polymer and a recognition entity. The composition may include, for example, a sol, a gel, a blend, particles, or a film. The recognition entity may be a nucleic acid recognition entity, a protein recognition entity, an aptamer, or the like.

In another set of embodiments, the article includes a microarray. The microarray includes a luminescent polymeric composition. The composition may include, for example, a particle, a sol, a blend, or a film. In another aspect, the invention comprises a sol or a blend including a luminescent polymer.

In another set of embodiments, the article includes a luminescent polymer a recognition entity, and an energy migration pathway between the luminescent polymer and the recognition entity. The recognition entity may be a nucleic acid recognition entity, a protein recognition entity, an aptamer, or the like. The article, in yet another set of embodiments, includes a substrate having a surface charge. In one embodiment, the substrate comprises a luminescent polymer.

In still another set of embodiments, the article is defined, at least in part, by a mixture including a first polymer having a first excitation wavelength and a first emission wavelength, a second polymer having a second excitation wavelength and a second emission wavelength at lower energy than the first emission wavelength, and an energy migration pathway between the first polymer and the second polymer. The mixture is able to emit light at substantially the second emission wavelength when incident light at the first excitation wavelength is applied to the composition.

In yet another set of embodiments, the article includes a composition comprising a luminescent polymer and an aptamer.

In yet another aspect, the invention is a method. In one set of embodiments, the method includes the steps of providing a homogeneous composition comprising a first polymer and a second polymer different from the first polymer, exposing the composition to energy that is substantially absorbed by the first polymer but is not substantially absorbed by the second polymer, and detecting light emitted from the composition, wherein the light is emitted substantially by the second polymer. The method, in another set of embodiments, is defined in part by the steps of providing an article comprising a luminescent polymer and a recognition entity, the article having a luminosity, allowing a molecule to bind to the recognition entity, and detecting a change in luminosity of the article. The recognition entity may be a nucleic acid recognition entity, a protein recognition entity (e.g., an antibody or lectin), an aptamer, or the like.

In one set of embodiments, the method is defined by the steps of providing a luminescent article, and a quenching agent prevented from interacting with the article, and allowing a nucleic acid, protein, or small molecule to facilitate interaction between the quenching agent and the luminescent article, causing quenching of the luminescent article under conditions in which, in the absence of the nucleic acid, protein, or small molecule, the quenching agent remains prevented from interacting with and quenching of the luminescent article. In another set of embodiments, the method includes allowing a nucleic acid molecule, protein, or small molecule to bind to a particle comprising a quenching agent, releasing the quenching agent from the particle, and allowing the quenching agent to bind to a luminescent article.

In another aspect, the invention includes a system. In one embodiment, the system includes a recognition article comprising a quenching agent and a recognition entity, and a luminescent article in fluid communication with the recognition article. The recognition entity may be a nucleic acid recognition entity, a protein recognition entity, an aptamer, or the like.

In still another aspect, the invention is directed to a method of making any of the embodiments described herein. In yet another aspect, the invention is directed to a method of using any of the embodiments described herein.

Other advantages, novel features, and objects of the invention will become apparent from the following detailed description of non-limiting embodiments of the invention when considered in conjunction with the accompanying drawings, which are schematic and which are not intended to be drawn to scale. In the figures, each identical or nearly identical component that is illustrated in various figures typically is represented by a single numeral. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

Non-limiting embodiments of the present invention will be described by way of example with reference to the accompanying drawings in which:

FIG. 1 illustrates chemical structures useful in the invention;

FIG. 2 illustrates a method of making a sol of the invention;

FIGS. 3A and 3B illustrate an embodiment of the invention having a charged surface;

FIGS. 4A and 4B illustrate another embodiment of the invention, having a nucleic acid recognition entity immobilized on a surface;

FIGS. 5A and 5B illustrate an embodiment of the invention having a nucleic acid with a stem-loop structure;

FIGS. 6A and 6B illustrate an embodiment of the invention having quenching agent;

FIG. 7 illustrates an embodiment of the invention able to be used in a microarray;

FIG. 8 illustrates an embodiment of the invention having an intercalating agent;

FIG. 9 illustrates spectra of certain embodiments of the invention;

FIGS. 10A and 10B illustrate data associated with an embodiment of the invention that is photostable;

FIGS. 11A-11D illustrate data associated with an embodiment of the invention and its associated spectra under certain conditions;

FIG. 12 illustrates a method of the invention;

FIG. 13 illustrates another method of the invention;

FIG. 14 illustrates yet another method of the invention;

FIG. 15 illustrates a nucleic acid probe of the invention;

FIGS. 16A and 16B illustrate quenching data for an embodiment of the invention able to bind to a nucleic acid sequence;

FIG. 17 is a photocopy of a fluorescent scan of various concentrations of a fluorescent dye on a microscope slide;

FIG. 18 is a photocopy of a fluorescent scan of a polymer-coated slide;

FIG. 19 is a photocopy of a fluorescent scan of a second polymer coated slide; and

FIG. 20 is a graph illustrating correlation data for the results shown in FIG. 19.

### DETAILED DESCRIPTION

U.S. patent application Ser. No. 09/997,999, entitled “Luminescent Polymer Particles,” by Hancock, et al., filed Nov. 30, 2001 is incorporated herein by reference in its entirety.

The present invention involves a series of articles, compositions, methods, and kits. Some aspects of the invention include articles such as particles, sols, blends, dispersions, films, or microarrays that comprise luminescent polymers, as well as methods for making and using such articles. In some cases, the luminescent polymer may be characterized in part by having a delocalized π-orbital structure, which can allow the polymer to have a high degree of luminosity. The polymers of this invention may also have, in some embodiments, bulky substituents to prevent intermolecular π-π interactions that can decrease luminosity. Some articles may include more than one luminescent polymer, for example, a first polymer that absorbs energy and directs the energy to a second polymer that releases the energy. The articles of the invention may be used, in certain embodiments, to detect the presence of other compounds such as single molecules, proteins, or specific nucleic acid sequences, as well as cells, bacteria, and viruses. In one set of embodiments, the luminescent polymers are associated with an entity that can interact with, for example, a nucleic acid or a protein. The association may be direct, or through an energy transfer pathway. The entity can be, for example, a nucleic acid, a charged surface, an intercalating agent, or an entity that releases a quenching agent that interacts with the luminescent polymer.

The following definitions will aid in understanding the invention.

“Derivative,” “chemical derivative,” “derivatizing,” and similar terms are given their ordinary meanings in the field of chemistry. A derivative may be any chemical substance structurally related to another chemical substance.

“R” generally refers to a hydrocarbon group (including cyclic hydrocarbon groups), optionally interrupted by hetero groups. As used herein, “hydrocarbon,” “alkyl,” and similar terms include alkyl, alkenyl, alkynyl, cycloalkyl, aryl, alkaryl, aralkyl, and the like. Examples of such hydrocarbon groups may include methyl, propenyl, ethynyl, cyclohexyl, phenyl, tolyl, benzyl, hydroxyethyl and the like. Hetero groups may include —O—, —CONH—, —CONHCO—, —NH—, —CSNH—, —CO—, —CS—, —S—, —SO—, —(OCH₂CH₂)ₙ— (where n may range from 1 to 10), —(CF₂)ₙ— (where n may range from 1 to 10), olefins, and the like. “Hydrocarbon,” “alkyl,” and similar terms may also include alcohols and hydrogen. By way of example, “R” may be an alkyl group, preferably having 1 to 24 carbon atoms, more preferably 1 to 18 carbon atoms; an alkenyl group, preferably having 2 to 4 carbon atoms; an alkylamino group, preferably having 1 to 8 carbon atoms, and optionally substituted on the nitrogen atom with one or, preferably two alkyl groups, preferably having 1 to 4 carbon atoms; an alkyl group, preferably having 1 to 4 carbon atoms, having a five- or six-membered heterocyclic ring as a substitutent; an allyloxyalkyl group, preferably having up to 12 carbon atoms; an alkoxyalkyl group, preferably having a total of 2 to 12 carbon atoms; an aryloxyalkyl group, preferably having 7 to 12 carbon atoms; an arylalkyl group, or the like.

A “dalton” (Da) is an alternate name for the unified atomic mass unit (grams/mole). The dalton is accepted by SI as an alternate name for the unified atomic mass unit. Similarly, a “kilodalton” (kDa) is 1000 daltons.

“Proteins” and “peptides” are well-known terms in the art, and are not precisely defined in the art in terms of the number of amino acids that each includes. As used herein, the term “protein” also includes peptides.

A “sample suspected of containing” a particular component refers to a sample with respect to which the content of the component is unknown. “Sample” includes both chemical samples and naturally-occurring samples, such as physiological samples from humans or other animals, samples from food, etc. Typical naturally-occurring samples may include saline, cells, blood, urine, ocular fluid, saliva, fluids, lymph nodes, needle biopsies, etc.

As used herein, “an aqueous solvent” and an “organic solvent” are given their ordinary meanings in the field of chemistry. An aqueous solvent is a solvent with a relatively high dielectric constant that includes water, and an organic solvent is a solvent with a relatively low dielectric constant. Additionally, as used herein, a “solvent of the opposite phase” refers to a solvent that is not miscible with, and phase-segregates from, a reference solvent, i.e. one having a dielectric constant different enough from the reference solvent to cause a phase segregation when the two are combined. For example, when referring to an organic solvent, a solvent of the opposite phase would be an aqueous solvent or a solvent having a high dielectric constant, and vice versa.

In a “luminescent” molecule, when a form of energy, such as a photon, interacts with a molecule, energy is absorbed by the molecule, allowing an electron to go from a lower energy state into an “excited” or higher energy state. The site within the molecule where the energy is absorbed may be referred to as the activation site. The energy absorbed by the molecule may be referred to as an “exciton.” Although an exciton is not a physical particle, it can be analyzed as though it were a particle located within the molecule. The absorbed energy later can be released as a photon, as the excited electron descends from the higher energy state to a lower state. One form of energy excitation is by the interaction of the molecule with an incident photon corresponding to visible light, ultraviolet light, or other electromagnetic frequencies, in which case the energy of the incident photon is termed the “excitation energy” or “excitation frequency.” However, other methods of excitation are also possible, such as through incident electrons, electrical current, friction, heat, chemical or biological reactions, the influence of sound waves, or other methods that are known or would be apparent to those of ordinary skill in the art.

The excited electron may descend to a lower energy state by one of two methods. During fluorescence an electron travels directly to the ground state, releasing a photon in the process. However, during phosphorescence, the electron descends to another excited state before releasing a photon and returning to the ground state. Typically, fluorescence occurs on a shorter time scale than does phosphorescence. Phosphorescence may occur for a considerable period of time after the excitation source has been withdrawn, up to several seconds or minutes, while fluorescence generally stops almost immediately after the excitation source has been withdrawn. Some materials may be fluorescent without being phosphorescent, or vice versa. The location within the molecule where the photon is emitted by the electron as it descends from the excited energy state is typically referred to as the chromophore of the molecule.

The photon emitted by the electron as it descends from the excited energy state may have any wavelength or frequency. This frequency or wavelength is typically referred to as the “emission frequency” or the “emission wavelength.” In certain cases, more than one photon may be emitted as the electron descends from the excited energy state. The electron may also descend to a lower energy state without releasing a photon, optionally followed by release of a photon. For example, conformation changes in vibrational energy (e.g., heat energy or kinetic energy), or other electronic rearrangements are avenues through which an electron can descend to a lower energy state without, or prior to, emitting a photon.

In one aspect, the present invention includes one or more luminescent materials. These materials may be inorganic or organic, for example, as in a polymer. In some embodiments, the luminescence of the material may be facilitated by an electronic structure caused by various means, such as by a coordinated metal, a bioluminescent or a chemiluminescent moiety, or a π-orbital structure. In some molecules, the luminescent properties may be facilitated by the presence of delocalized π (pi)-orbital structures within the molecule; of course, not all luminescent materials require an extended π-orbital structure to achieve luminescence, for example, poly(vinyl carbazole). Typically, in a delocalized π-orbital structure, the electrons in the p-orbitals forming the π bond cover multiple atom centers, which are said to be in “π-electron communication” or “π-communication.” As used herein, “π-orbitals,” “π structures,” “π-backbone,” and the like are given their ordinary definitions as is understood in organic and inorganic chemistry, where the electrons in π-oriented orbitals between adjacent atoms are shared, creating a chemical bond between the adjacent atoms. Similarly, “π-stacking” or “intermolecular π-π interactions” generally refers to structural interactions between two or more molecules in which the π-orbitals of nearby molecules are adjacent, overlapping, or otherwise affect each others' properties, for example by interfering with the release of photons causing a decrease in luminosity. In some cases, the luminescent polymers may be selected, for example, to have a certain excitation or emission wavelength. Non-limiting examples of luminescent polymers include poly(phenylene ethynylene), poly(phenylene vinylene), poly(p-phenylene), poly(thiophene), poly(pyridyl vinylene), poly(pyrrole), poly(acetylene), poly(vinyl carbazole), poly(fluorenes), and the like, as well as copolymers and/or derivatives thereof.

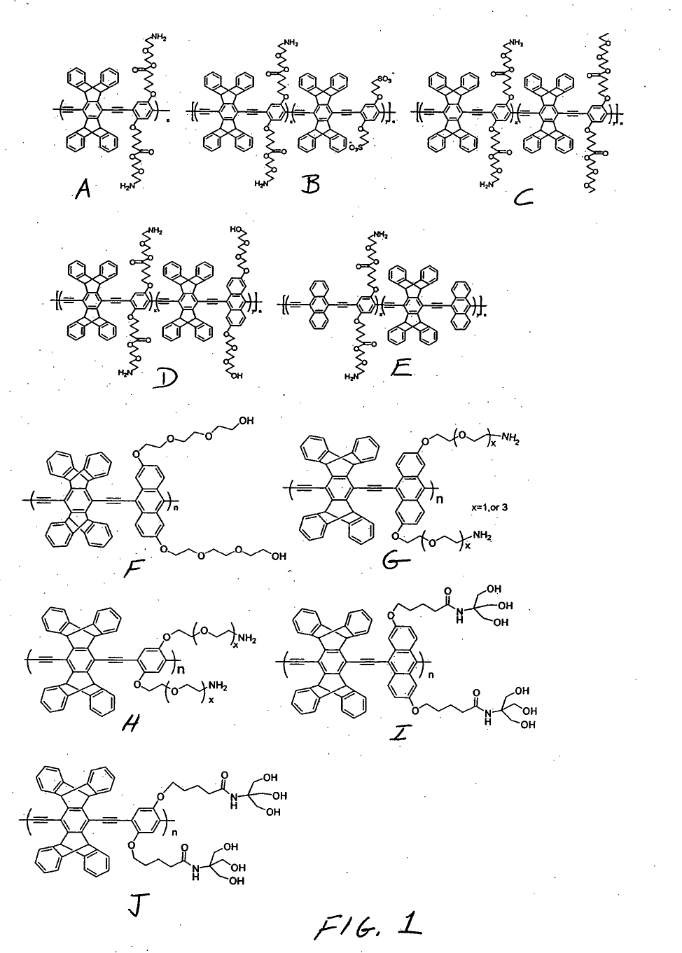

FIG. 1 illustrates example luminescent polymers. In this figure, the example luminescent polymers incorporates pentiptycene subunits in a poly(phenylene ethynylene) backbone. In some embodiments, the polymer may include more than 1 type of repeat unit, as illustrated in FIGS. 1B, 1C, 1D, and 1E. The two or more repeat units may be present at any ratio. In some embodiments, the repeat units may alternate along the polymer chain; in other embodiments, they may form linear blocks of constant or variable length; and in still other embodiments, the repeat units may be randomly sequenced. For example, if two repeat units are present in a polymer, the repeat units may be at a ratio of 2:3, 1:3, 1:1, 2:1, 4:1, etc. In some embodiments, other subunits may also be incorporated within the polymer, for example, to alter the emission wavelength of the polymer. For instance, FIGS. 1D and 1E have anthracene subunits incorporated within the polymer backbone.

In one set of embodiments, the luminescence of the polymer may be facilitated by a delocalized π-orbital. Delocalized π-orbitals may exist in a variety of structures, including but not limited to double bonds, triple bonds, benzene rings, naphthalene rings, anthracene rings, pyridines, carbazoles, iptycenes, and the like. Other aromatic systems having similar arrangements of atoms to produce delocalized π-bonds are also within the scope of the invention, as well as moieties containing delocalized π structures having additional substituents, such as oxygen, sulfur, nitrogen, a halogen, or the like. For example, nitrogen atoms may often be substituted for carbon atoms within a delocalized π structure, such as in pyridines and similar compounds.

When a quantum of energy is absorbed by the polymer, the resulting exciton may be transmitted along “an energy migration pathway” of the polymer, in a process referred to as “intrachain jumping.” For example, an electron may be transferred along a π-orbital backbone due to the presence of one or more delocalized π-orbitals. The π backbone, or other analogous structure(s) able to transmit excitons, typically defines the energy migration pathway. In some cases, the π backbone may amplify the effect of the absorbed energy quanta, as the effects of the exciton can be transmitted along the energy migration pathway to more than one chromophore. Besides intrachain transmission, the exciton can, in some cases, be transmitted between different polymer molecules, or between a polymer molecule and a non-polymer molecule, in a process referred to as “interchain jumping,” for example, when the molecules have overlapping π-orbitals.

In some embodiments of the invention, a substantial amount of interchain jumping may occur between different polymer molecules. For example, incident light may excite a first polymer molecule, creating an exciton in that molecule. The incident light may be of a frequency that is unable to create an exciton within a second molecule proximate to the first polymer molecule, for example, a second polymer molecule. However, due to interchain jumping, the exciton may be transferred from the first to the second molecule. The second molecule may then emit the exciton as a photon. The degree of interchain jumping may be substantial in some cases, such that 50%, 70%, 90%, or 95% or more of the excitons created in the first polymer molecule is transmitted to the second molecule, and are not emitted as photons by the first polymer molecule. In one set of embodiments, the first polymer molecule may transfer the exciton to a second molecule that is non-luminescent, for example, a chemical, biochemical, or biological molecule such as a nucleic acid recognition entity. In one set of embodiments, the polymers are used in a fluorescence resonance energy transfer (FRET) system.

The transfer of an exciton may occur by any suitable means, for example, transmission through the release of a photon from one molecule and the absorbance of that photon by another molecule or the same molecule, transmission through the transfer of kinetic energy, transmission due to the overlap of π-orbitals between the different molecules, or longer range energy transport through Dexter or resonant energy transfer mechanisms. π backbone structures may be used, for example, to amplify the sensitivity of the polymer to external compounds.

The luminescence of polymer molecules of the invention may also be modified or controlled by adding certain substituents to the polymer. For example, the luminescence or emission wavelength of photons emitted by a poly(phenylene ethynylene) particle may be controlled or altered through chemical substitution along the poly(phenylene ethynylene) polymer backbone, or along side chains attached to the backbone. The substituents may have any ratio or distribution within the molecule. In some embodiments, the substituents have a delocalized π-orbital structure, or the substituents alter the energy or frequency of emission from excitons that propagate along the polymer. In other embodiments, the substituents may induce π-orbital delocalization within the molecule. For instance, in some embodiments, the additional substituent may include a benzene ring or other aromatic group, such as an anthracene moiety. For example, the substituent may be an anthracene unit joined to the polymer backbone through a 9,10 linkage, where the substituent is one of a number of repeat units of the polymer.

In some embodiments, individual polymer molecules may be prevented from aggregating with each other, or interacting with each other through π-orbital overlap, for example, by the distribution of charges within each polymer, by the presence of bulky substituents within each polymer, or due to the physical properties of the particles containing the individual polymer molecules. In some embodiments, the composition may include single, isolated polymer molecules. For example, if the article is a dispersion of polymers having a sufficiently high molecular weight, the dispersion may include nanometer-sized isolated polymer molecules dispersed within the solvent.

One aspect of the present invention involves the prevention of π-stacking in polymers through the use of one or more “bulky” monomers that prevent adjacent or nearby neighboring molecules from touching or interacting with each other. This may be done, for example, to prevent intermolecular π-π interactions from occurring that cause the luminosity of the polymer to decrease. For example, two adjacent or nearby molecules, having delocalized π-orbitals, can interfere with the release of photons from each molecule. Also, under certain circumstances, one polymer molecule may self-interfere. As the interference may be caused by interacting π-orbitals, in one set of embodiments, the polymers of the present invention may include bulky monomers or substituents defined by any chemical moiety able to keep nearby polymer π-orbitals separated. For example, the bulky group(s) may comprise surfactants, proteins, or organic molecules. In some instances, the bulky group(s) may comprise a pentiptycene, a triptycene, or other iptycene and iptycene-related moieties.

In some embodiments, a bulky group may have a smallest dimension of no less than about 0.25 nm, where the “smallest dimension” may be defined as the smallest dimension of the smallest imaginary box able to contain the bulky group. In other embodiments, the smallest dimension can be less than about 0.30 nm, 0.35 nm, 0.40 nm, 0.45 nm, 0.50 nm, or 0.60 nm. The bulky group may be located anywhere within the polymer. For example, the bulky group may be adjacent to or be part of the backbone of the polymer. The bulky group may also be attached to the polymer chain through the use of pendant groups connected to the backbone of the polymer, or be randomly distributed within the polymer. In some cases, the bulky group may include delocalized π-orbital structures, such as double bonds, triple bonds, benzene moieties, anthracenes, pyridines, carbazoles, or the like. In certain embodiments, the bulky group may include several benzene or other aromatic rings, forming a bicyclic or other multicyclic structure, for example, as in a pentiptycene moiety.

Additional chemical groups or functionalities may be attached to, or form part of, the bulky group. If the chemical group or functionality is attached to the bulky group, the chemical group or functionality may be attached by any suitable means, such as through ionic, covalent, or hydrogen bonds. The chemical groups may, for example, provide additional chemical functionalities, assist in polymer separation, or assist in the dissolution, suspension, or dispersion of the polymer in the surrounding fluid or other media.

By minimizing the intermolecular π-π interactions between nearby or adjacent polymers, the shape of the luminescence emission spectra may not change significantly as the environment of the polymer molecule is changed, for example, from an organic phase to an aqueous phase or vice versa. The polymers may thus remain luminescent while the polymer is incorporated into an article such as a particle, a sol, or a dispersion. The emission spectra of the particles may not necessarily be dependent on the size of the particle or the environment that the particle is located in. Thus, for example, the polymer may be luminous when dissolved in an organic solvent or an aqueous solvent. The luminosity of the particles may not decrease, or the spectra of the particles may not shift significantly, after at least one day, preferably at least one week, more preferably at least one month, more preferably at least two months, or still more preferably, at least three months or longer. In some embodiments, the luminosity may remain substantially constant for an indefinite period.

In one set of embodiments, the luminescent polymer includes a bicyclic or other multicyclic ring system, for example, as in an iptycene moiety or a related molecular structure. An iptycene moiety generally has a [2.2.2]bicyclic ring system, formed from the intersection of geometric planes, for example, as defined by aromatic rings fused within the [2.2.2]bicyclic ring system (“arene planes”). The aromatic rings on each of the branches of the [2.2.2]bicyclic ring system, may be connected to, for example, another [2.2.2]bicyclic ring system, or a delocalized π-orbital structure, such as a double bond, a triple bond, or an aromatic group. In one embodiment, the polymer comprises a structure: 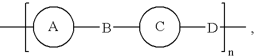
where n is at least 1, at least one of A and C comprises a bicyclic ring system, and at least one of B and D comprises a triple bond. In yet another embodiment, the polymer is a copolymer formed from a plurality of monomers, where at least one monomer comprises a structure: 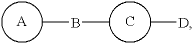
where at least one of A and C comprises a bicyclic ring system, and at least one of B and D comprises a triple bond. Those of ordinary skill in the art will recognize that additional syntheses may result in any of a wide variety of molecules useful in the present invention, for example using a diene or a dienophile.

In one set of embodiments, a polymer of the invention may include a backbone chain. The longest chain of covalently bonded atoms within the polymer is typically defined to be the backbone. The backbone may have other substituents attached to it or interspersed with it, for example, additional polymer chains (e.g., as in a copolymer), or other species or derivatives, for example, one or more poly(ethylene) oxide chains. The backbone may also include interspersed rings of atoms, such as, for example, benzene rings, as long as the overall structure of the backbone is continuous. In certain embodiments, the backbone may include various delocalized π structures such as aromatic groups, or double or triple bonds. In one embodiment, the backbone includes a π backbone.

In embodiments where the polymer is composed of more than one monomer type (i.e., as in a “copolymer”), the monomer types forming the copolymer may be arranged in any fashion. It is to be understood that in any embodiment employing a polymer, the polymer being employed may be a copolymer in some cases. Each of the monomer types within the copolymer may also be referred to as a “repeat unit.” In addition, various branches off of the copolymer backbone are also possible. For instance, one or both of the monomer types may be branches, forming a blocky branched scheme. The backbone in other embodiments may consist of one polymer type, and a second polymer type may branch off the first polymer type, forming a grafted copolymer. The branches may be randomly distributed on the copolymer backbone or they may be regularly situated on each monomer in some cases, forming a side chain-modified copolymer. Other arrangements of the monomer types within the copolymer are also within the scope of the present invention. Additional monomer types (e.g., as in a terpolymer having three or more repeat units, or a higher order copolymer having multiple repeat units) may also be distributed within the polymer in any fashion, for example, as in an alternating fashion, a random fashion, or a block fashion. Additional non-monomer groups can also be attached to the polymer at any position, for example, at one or more termini of the copolymer, as a substitute for one of the monomers, or attached to a side chain of the monomer.

The monomer types may have any distribution ratio. For example, in one embodiment, there may be two monomer types, having a 50:50 ratio. In other embodiments, the ratio between the two monomer types may be 1:2, 1:3, 1:5, 1:10, 1:50, 1:100, 1:1000, or 1:10,000. Any monomer type may be the most prevalent monomer type. Other ratios of the monomer types may also be possible. For example, in a triblock polymer, there may be three monomers having any distribution ratio, for example, 1:1:1, or 1:2:3.

The polymer molecule may have any size or molecular weight. In some cases, the molecule may consist of at least 5 monomers. In other cases, the molecule may have at least 10 monomers, 50 monomers, 100 monomers, at least 1000 monomers, at least 10,000 monomers, at least 100,000 monomers, or at least 1,000,000 monomers. The polymer may also have any molecular weight, for example, at least about 100 daltons. In other embodiments, the polymer may have a molecular weight of at least about 1000 daltons, at least about 10,000 daltons, at least about 100,000 daltons, or at least about 1,000,000 daltons. In some cases, the size of the molecule may approach nanoscopic dimensions. For example, a single polymer molecule can have a mean diameter of at least about 5 nm, corresponding to a very high molecular weight. In other cases, the diameter of the polymer molecule may be at least about 10 nm, at least about 20 nm, at least about 50 nm, at least about 100 nm, or at least about 1 μm.

The polymer molecules of the invention may have any size distribution. For example, the polymer molecules may have a very narrow distribution, where most of the molecules have a single size or a very small range of sizes. The size distribution of the polymer molecules can also be very broad in some cases. If the polymers are incorporated into particles, the polymers in the particles may have the same or different molecular weights. The size of the particle also may not be directly related to the size of the polymer molecules contained therein.

The polymers of the invention may be formed by any suitable technique. For example, various monomer units may be combined together to form the polymer. These may include, for example, iptycene complexes, pentiptycene complexes, poly(ethylene oxide)s, tetrasubstituted benzenes, disubstituted benzenes, or the like. Monomer units having delocalized π-electron configurations can also be included, such as those including double bonds, triple bonds, aromatic groups, pyridines, carbazoles, anthracenes, and the like. Various moieties located on the monomers may be used during polymer synthesis, for example, halogens or acetylides.

The monomer units may be reacted to produce the polymers of the invention by any suitable technique. For example, various monomers can be reacted together over a palladium catalytic complex to produce a polymer. In another embodiment, the polymer is produced by a ring-opening synthesis technique, such as in a ROMP (Ring Opening Metathesis Polymerization) reaction. In still other embodiments, a hydroquinone is reacted to produce a dialkoxy dihalogenated benzene ring for use as a monomer in a polymerization reaction. The dialkoxy group may include a charged entity, such as a sulfonate group.

The polymer may be hydrophobic or soluble in organic solvents, such as toluene, tetrahydrofuran, chloroform, dimethylformamide or methylene chloride. In certain embodiments, the polymer possesses anionic sulfonate pendent groups attached to the backbone or to each other, allowing the polymer to be hydrophilic or soluble in polar solvents, such as water or alcohols, for example, isopropanol, ethanol, or methanol. In certain embodiments of the invention, the polymer may be a luminescent polymer amphiphile (attracted to both organic and aqueous environments), or it may possess a luminous polymer backbone.

In one set of embodiments, a binding site is associated with the luminescent polymer. The binding site may comprise a recognition entity able to bind to a biological, biochemical or chemical molecule in solution (the “analyte”). As used herein, a “recognition entity” is an entity that is able to specifically recognize another molecule, for example, a protein, a peptide, a nucleic acid, a small molecule, etc. In some cases, less than about 1 pg/g, preferably less than about 100 fg/g, more preferably less than about 10 fg/g, and still more preferably, less than about 1 fg/g of analyte may be detected by the invention. In one embodiment, the recognition entity may comprise a nucleic acid recognition entity; in another embodiment, the recognition entity may comprise a protein recognition entity or an aptamer. The recognition entity may be attached to the luminescent polymer, for example, by covalent attachment, ionic coordination, charge interactions, and/or nonspecific hydrophobic adsorption. As one example, the recognition entity may be cross-linked to the luminescent polymer or to the article containing the luminescent polymer, for example, by ultraviolet crosslinking.

A “nucleic acid recognition entity,” as used herein, is an entity able to recognize and/or specifically bind to a nucleic acid, for example covalently, or through non-covalent interactions such as hydrogen or van der Waals interactions. For example, the nucleic acid recognition entity may be a nucleic acid such as RNA, DNA, PNA (peptide nucleic acid), a grip nucleic acid, or combinations thereof (e.g., a chimeric nucleic acid), a protein able to bind to a nucleic acid such as an endonuclease or a nucleosome; an intercalating agent such as ethidium bromide, propidium bromide, acriflavine, acridine orange, or the like. If the nucleic acid recognition entity is a nucleic acid, the nucleic acid may have any configuration, for example single-stranded, double-stranded, a stem-loop structure, a partially single-stranded and partially double-stranded configuration, etc. In certain embodiments, the nucleic acid recognition entity may be positively charged, or uncharged in some cases. In some embodiments, the nucleic acid recognition entity may be able to specifically recognize a particular sequence of bases within the target nucleic acid. For example, the nucleic acid recognition entity may specifically recognize a sequence of five bases, 10 bases, 30 bases, 100 bases, 300 bases, 1000 bases, or more in some cases.

As used herein, a “protein recognition entity” is an entity able to recognize and/or bind to a protein or a peptide, for example, covalently or through non-covalent interactions such as hydrogen or van der Waals interactions. For example, the protein recognition entity may be a protein, an enzyme, an aptamer, an antibody, lectin, or the like. As used herein, an “aptamer” is a nucleic acid sequence (e.g., DNA or RNA) able to specifically bind to a protein, a peptide, or a small molecule (e.g., a molecule having a molecular weight of less than about 1000-1500 Da). A “recognition entity” generically includes a nucleic acid recognition entity, a protein recognition entity, an aptamer, etc.

“Specifically bind” and similar terms are given their ordinary meanings as used in biochemistry, generally including specific recognition between the two entities to be bound, but excluding non-specific binding. For example, specific binding can occur between two nucleic acid strands having complementary sequences, a protein or an enzyme adapted to recognized a specific nucleic acid sequence or a peptide, a nucleic acid sequence adapted to recognize a protein (e.g., as in an aptamer), a charge or a series of charges that specifically bind a nucleic acid sequence, etc. In one set of embodiments, the article may be a charged surface or a charged polymer. The charge may be created by any suitable technique, for example, via electric or electrostatic interactions, or due to charged moieties present within or on the article. Similarly, “non-specific binding” and similar terms, as used herein, are given their ordinary meanings in biochemistry.

The binding of an analyte to a recognition entity may be determined by any suitable technique. For example, a luminescent polymer, having one or more nucleic acid molecules and/or recognition entities such as protein recognition entities, aptamers, and/or nucleic acid recognition entities attached thereon, may be detected via a fluorescent tag in a FISH assay (fluorescence in situ hybridization). As used herein, the term “determining” generally refers to the analysis of a species, for example, quantitatively or qualitatively, or the detection of the presence or absence of the species. “Determining” may also refer to the analysis of an interaction between two or more species, for example, quantitatively or qualitatively, or by detecting the presence or absence of the interaction. Example techniques include, but are not limited to, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; turbidity measurements; or PCR (polymerase chain reaction) systems.

In one aspect of the invention, a surface including a luminescent polymer also includes a recognition entity able to bind an analyte, e.g., a nucleic acid, a protein recognition entity, an aptamer, etc. The luminescent polymer may be present in any form, for example, as an integral part of the surface (for example, if the surface is a polymer or a copolymer), or bound to the exterior of the surface. The surface may be any suitable surface that can be used to bind the analyte. For example, in one embodiment, the surface is the surface of a film. In other embodiments, the surface is the surface of a particle (which may be porous), a sol, a blend, or the like. As one example, if particles are used, the particles may be used in any system where the detection of an analyte such as a nucleic acid is desired. For instance, the particles may be used in a flow system, where they are passed from one region to another region (for instance, across a region where they are exposed to a nucleic acid or a protein); the particles may be immobilized (for example, on a substrate such as the wall of a capillary tube, or on a membrane) and the nucleic acid or protein passed over them; the particles may be added to a cell culture to detect free nucleic acid or protein; etc.

In one set of embodiments, the recognition entity is a surface that is charged or contains charged groups, preferably in such a way as to attract an analyte such as a nucleic acid, a protein, or a specific sequence. For example, a negatively-charged nucleic acid may be attracted to a surface containing a certain positive charge distribution. In one embodiment, the analyte may interact with specific charge groups present on the surface. The binding of the analyte to the surface may be detected, for example, by a change in luminosity of a surface including luminescent polymer, for example, by an increase or decrease in luminosity. The increase or decrease in luminosity can be significant in some cases, with changes in luminosity of at least one, two, three, or four orders-of-magnitude.

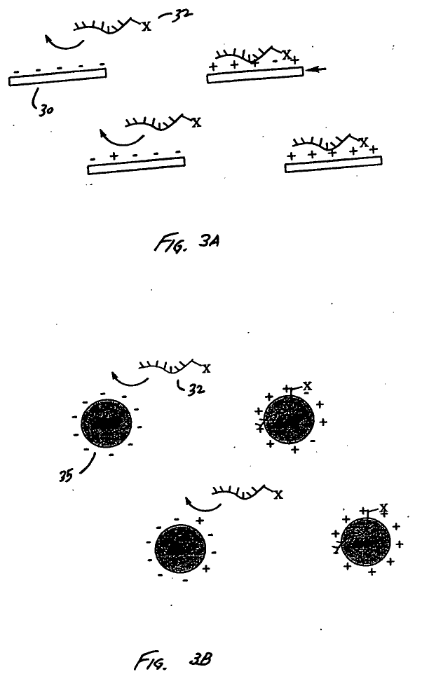

In one embodiment, the analyte is “tagged,” for example, with a quenching agent that can interact with the luminescent polymer. Binding of the analyte to the surface allows the quenching agent to interact with the luminescent polymer, thereby causing a decrease in the luminosity. Of course, in other embodiments, the analyte may be unlabeled; for example, binding of the analyte to the surface may cause a quenching agent to be released from the surface, allowing an increase in luminosity that can be detected. As an example, FIG. 3A illustrates a charged planar surface 30 and nucleic acid 32 that is to be detected. Nucleic acid 32 may be unlabeled or “tagged” in some fashion, for example, with a radioactive entity, a fluorescent tag, a quenching agent, or an unnatural base, represented in FIG. 3A with an “X.” The ability of nucleic acid 32 to bind to surface 30 may be a function of the charge or the charge distribution on surface 30. For example, nucleic acid 32 may bind to planar surface 30 if the planar surface has a certain charge and/or charge distribution. FIG. 3B illustrates a similar example, in which surface 30 is actually the surface of particle 35.

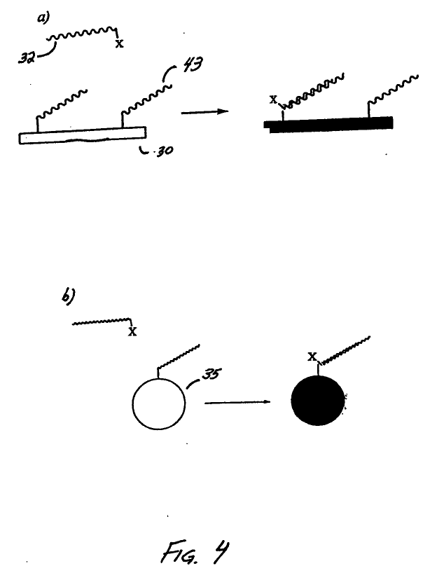

In another set of embodiments, the surface may include a sequence or structure complementary to the analyte, for example, the surface may include a nucleic acid sequence that is complementary to the target nucleic acid sequence. The sequence or structure may be bound to or otherwise immobilized on the surface, for example through the use of a coupling agent. For example, in FIG. 4, nucleic acid recognition entity 43 bound to surface 30 is a sequence of nucleic acids that is substantially complementary to a target nucleic acid 32 (i.e., a sequence that is able to specifically recognize the target nucleic acid, even if the sequences are not 100% complementary), optionally carrying a tag (indicated by an “X”). The binding of the nucleic acid to the nucleic acid recognition entity sequence may be detected, for example, by a change in luminosity of surface 30 that includes a luminescent polymer (e.g., an increase or decrease in luminosity). For example, in one set of embodiments where the surface includes a luminescent polymer, the target analyte may be tagged or otherwise labeled, for example, with a quenching agent that can quench the luminescent polymer. Binding of the analyte with the quenching agent to the recognition entity may cause a decrease in luminosity. In other embodiments, the analyte may be unlabeled; for example, binding of the analyte to the surface may cause a quenching agent to be released from the surface, allowing an increase in luminosity to be detected. FIG. 4B illustrates an example similar to FIG. 4A, in which surface 30 is actually the surface of particle 35.

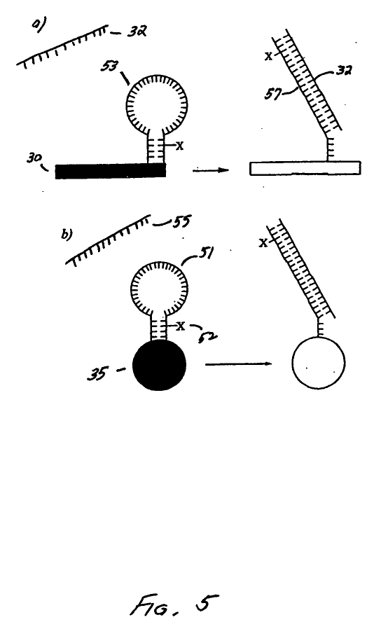

In one embodiment, the sequence complementary to the analyte may be a nucleic acid having a stem-loop structure. This structure may be bound to or otherwise immobilized on a surface including a luminescent polymer. For example, in FIG. 5A, a nucleic acid recognition entity having a stem-loop structure 53 is bound to surface 30. In some cases, a label such as a quenching agent may be bound to the base of the sequence, shown as an “X” in FIG. 5A; in this case, the quenching agent may interact with surface 30, decreasing its luminosity. Of course, other types of labels may also be used, for example, labels that can decrease, shift or enhance the luminosity of surface 30; or the nucleic acid may be free of labels. In FIG. 5A, upon the binding of nucleic acid 32, at least a portion of the stem-loop sequence of nucleic acids is released from surface 30, converting the nucleic acid strand from a stem-loop structure to structure 57 which specifically binds nucleic acid 32, moving the quenching agent away from surface 30. The movement of the quenching agent away from the surface may cause a change in luminosity, which can be detected or quantified. FIG. 5B illustrates a similar example, in which surface 30 is actually the surface of particle 35.

In another set of embodiments, a series of recognition entities is used, for example, in a method to detect or quantify a particular nucleic acid sequence, a small molecule, or a protein. For example, the surface of an embodiment of the invention may be the surface of a microarray, such as a protein microarray, a carbohydrate microarray, or a DNA or RNA microarray. The microarray may include a series of “spots” (i.e., locations), where each spot may have a recognition entity that can be associated with a luminescent article (e.g., a luminescent nanoparticle or a luminescent film). The spots may include one or more recognition entities or recognition entity types (e.g, protein and small molecule), which may be the same or different. In some embodiments, the recognition entities may be bound to or immobilized on the microarray; in other embodiments, the recognition entities may be in solution. The luminescent article may be part of the microarray (for example, a film or a blend), the luminescent article may be bound to or coated on the microarray, or the luminescent article may be separate from the microarray (e.g., a luminescent particle). In certain embodiments, the luminescent article may be associated with, or bound to, the recognition entity. The addition of an analyte such as a nucleic acid or a protein to each spot may cause a change in luminosity of the luminescent article, for example using one of the methods previously described, in spots containing a complementary recognition entity. In some cases, the change in luminosity may be used to determine the amount or degree of binding or specificity of an analyte to its corresponding recognition entity.

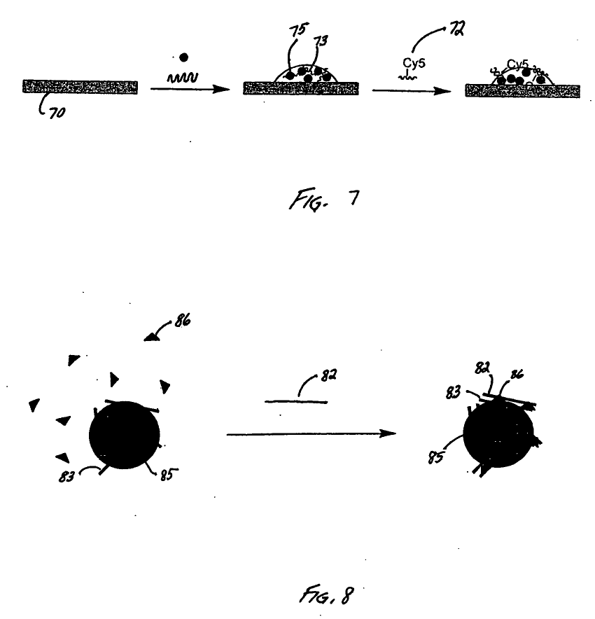

In the example illustrated in FIG. 7, luminescent particles 75 and nucleic acid recognition entities 73 are added to a spot on a microarray surface. Nucleic acid 72 labeled with Cy5, which, in this example, acts as a quenching agent, is then added to the spot. Nucleic acid 72 binds to nucleic acid recognition entities 73, which interact with and cause quenching of luminescent particles 75. In contrast, the addition of a nucleic acid labeled with Cy5 to a spot that does not contain nucleic acid recognition entities 73 specific to nucleic acid 72 may not result in the quenching of luminescent particles 75.

Quenching agents useful with the invention may include, for example, N-methyl dinitrobenzene morpholine, or biological dyes, such as Cy3, Cy5, dabcyl, dabsyl, BHQ-1, BHQ-2, dinitrophenol, or the like. The quenching agent may be associated with or separate from the luminescent polymer. The quenching agent may be in solution, or bound to a substrate such as a film or a particle. In certain embodiments, the quenching agent can quench the luminosity of the polymer with a Stem-Volmer constant of at least 10⁶ M⁻¹, preferably at least about 10⁷ M⁻¹, and still more preferably about 10⁸ M⁻¹. The magnitude of the Stem-Volmer constant and the sensitivity may vary as a function of solvent composition. The quenching agent may have its greatest quenching ability, as measured by the Stern-Volmer constant, at a specific ratio between the organic solvent and the aqueous solvent.

The use of quenching agents able to quench the luminescence of the polymers at high quenching rates may allow the polymers of the invention to have sensitive detection abilities. For example, an analyte, upon binding to a binding site connected to a quenching agent, may cause the quenching agent to be released from the luminescent polymer. The luminescent polymer, upon release of the quenching agent, may become highly luminescent, and this change may be easily detected. As another example, an analyte, upon binding to a recognition entity, may cause a quenching agent to be released or exposed. The quenching agent may then bind to a luminescent polymer. The binding of the quenching agent to the luminescent polymer may cause the luminescent polymer to lose its luminescence, which may then be detected.

As the specificity between the quenching agent and the polymer may be high, as measured by the Stem-Volmer constant, in some cases, a small number of binding events of an analyte to a recognition entity may lead to a large change in luminosity. For example, polymers containing conjugated π-backbone systems with one or more recognition entities may have a high degree of sensitivity, as one binding event may alter the π-backbone electron configuration or energy level, which may affect the luminescence of the entire polymer. Thus, in certain embodiments of the invention, the binding of less than 25 molecules, preferably less than 10 molecules, more preferably less than 7 molecules, more preferably still less than 5 molecules, and most preferably only 1 molecule, upon binding of the molecule or molecules to the recognition entity may cause a detectable change in the luminosity of the polymer associated with the recognition entity. Thus, in certain embodiments of the invention, a luminescent polymer may be used to detect a single analyte molecule in solution, for example, a protein, or a nucleic acid such as DNA or RNA.

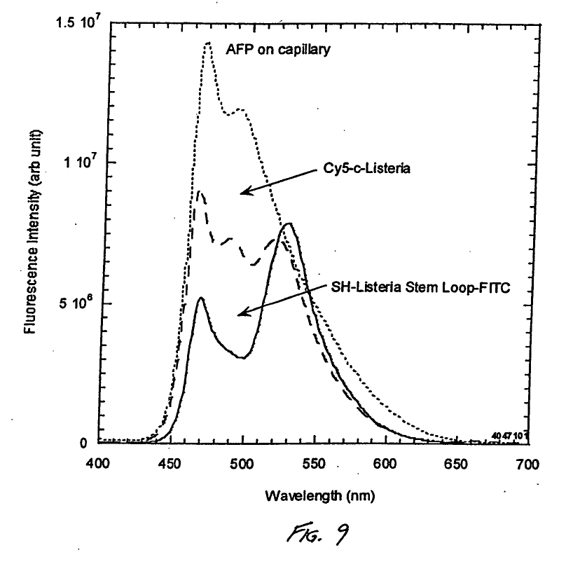

In one set of embodiments, an intercalating dye may be used as the quenching agent. The intercalating dye may interact with a target nucleic acid, for example, by binding to the hybridized nucleic acid, which may then cause a change in the luminosity of a luminescent polymer associated with the intercalating dye. For example, in FIG. 8, intercalating dye 86 is not able to interact with a single-stranded nucleic acid, but it can interact with a double stranded nucleic acid. The intercalating dye is in fluidic communication with a luminescent article, which is shown as a luminescent particle 85 in FIG. 8. Associated with luminescent particle 80 is nucleic acid recognition entity 81, which, in this example, is a sequence of nucleic acids complementary to a target nucleic acid 82. The addition of a nucleic acid 82 that is recognized by the nucleic acid recognition entity causes double-stranded nucleic acid to form, to which the intercalating dye is able to bind to. The association of the intercalating dye with the luminescent article then may cause a detectable change in the luminosity. In some cases, exciton interaction between the intercalating agent and the luminescent polymer may result in an increase in the luminosity of the intercalating agent. For example, incident light or other energy may excite the luminescent polymer, which can then collect and transmit excitonic energy to the intercalating agent, thus allowing the intercalating agent may have substantially higher luminosity.

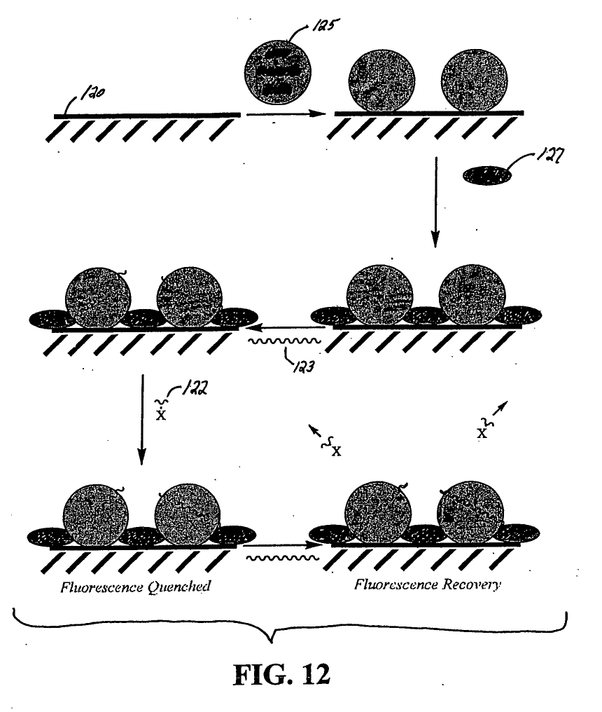

Other embodiments are illustrated in FIGS. 12-14. In the example illustrated in FIG. 12, particles 125 containing luminescent polymer are attached to surface 120 using any suitable technique, for example, through covalent or noncovalent bonding (of course, in other embodiments, the particles may be suspended in solution and not attached to a surface). Particles 125 may be, for example, sol particles or particles in a dispersion. Optionally, a blocking agent 127 may be used to reduce non-specific binding of the surface. The blocking agent may be a protein such as bovine serum albumin (BSA), a surfactant, a hydrophilic polymer (e.g., poly(ethylene glycol), or a nucleic acid such as a random oligonucleotide sequence. The particles may then be exposed to a nucleic acid recognition entity 123, for example, a complementary nucleic acid. In some cases, the nucleic acid recognition entity may be bound to the particles, for example, by exposure to ultraviolet light, causing ultraviolet crosslinking between the nucleic acid recognition entity and the particle. The nucleic acid 122 to be detected is then added. In some embodiments, for example, as illustrated in FIG. 12, the nucleic acid may be labeled, for example, with a quenching agent. If specific binding between the nucleic acid and the nucleic acid recognition entity occurs, then the quenching agent may interact with the luminescent polymer, causing a decrease in the luminosity of the luminescent polymer. In contrast, if no specific binding between the nucleic acid and the nucleic acid recognition entity occurs, then the luminosity of the luminescent polymer may not substantially decrease.

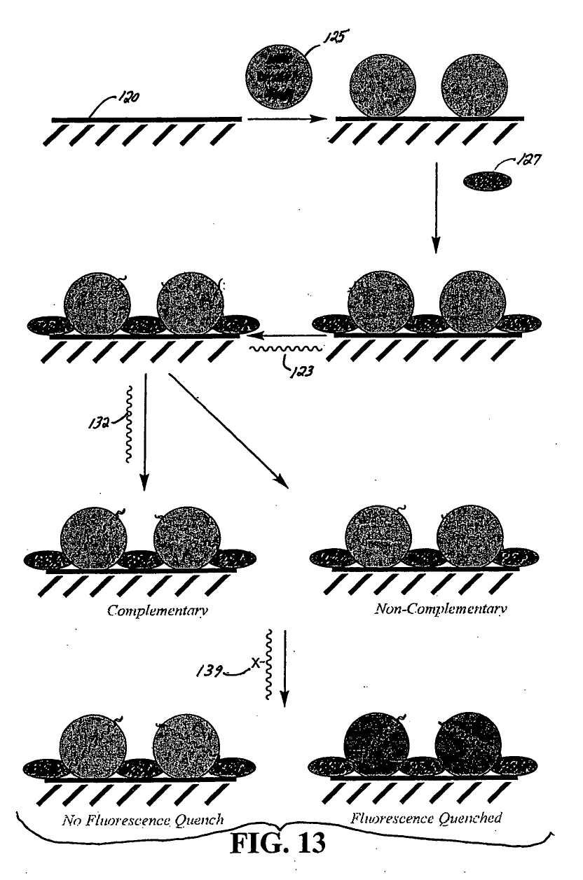

In other embodiments, the nucleic acid may be unlabeled. For example, in the particular embodiment illustrated in FIG. 13, unlabeled nucleic acid 132 is added to particles 125. Nucleic acid 132 substantially binds to complementary nucleic acid recognition entities but not to nucleic acid recognition entities that are not complementary. A developer 139, containing a label such as a quenching agent, is then added. The nucleic acid and the developer may be competitive for the nucleic acid recognition entity. If nucleic acid 132 already bound to the nucleic acid recognition entity, then developer 139 is unable to bind and thus, no quenching of luminescent particle 125 can occur; conversely, if nucleic acid 132 is not present, then developer 139 is able to bind to luminescent particle 125 and quenching may occur.

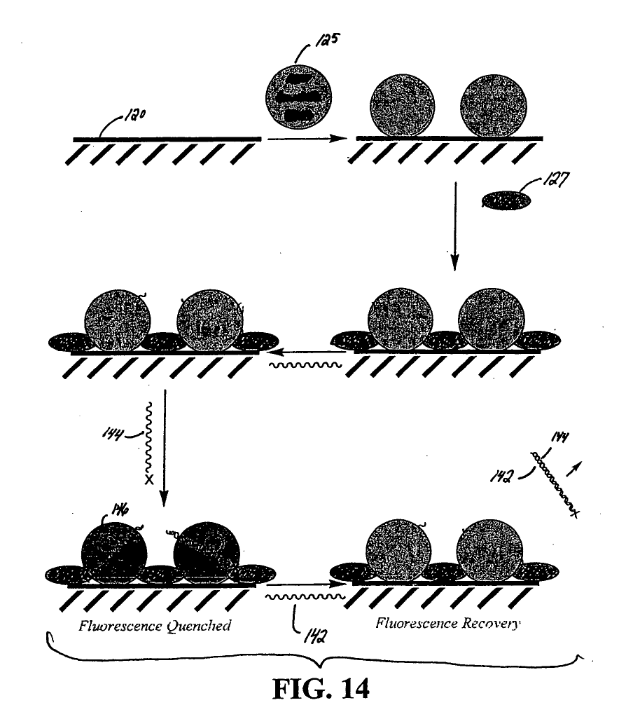

In still other embodiments, the nucleic acid may not directly interact with the luminescent molecule. For example, in the particular embodiment illustrated in FIG. 14, a nucleic acid recognition entity 144, such as a complementary nucleic acid, is bound to a nucleic acid immobilized on luminescent particle 125. In this embodiment, nucleic acid recognition entity 144 is substantially bound to the luminescent particle 125, although a mismatch target region 146 of the entity is not bound to the luminescent particle. The mismatch target region 146 may include a label such as a quenching agent. In this embodiment, a portion of nucleic acid recognition entity 144, for example, mismatch target region 146, is complementary to the nucleic acid to be detected 142. Binding of nucleic acid 142 causes release of nucleic acid recognition entity 144 from luminescent particle 125. The release of nucleic acid recognition entity 144 may then cause a change in the luminosity of luminescent particle 125.

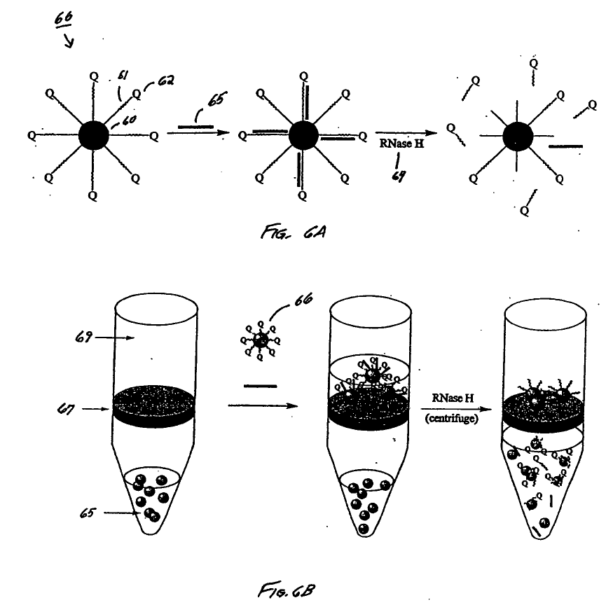

In another set of embodiments, the recognition entity is not directly associated with the luminescent polymer; the binding of an analyte to a recognition entity may cause the production of a signal able to interact with the luminescent polymer. Any signal able to interact with the luminescent polymer may be produced, for example, an electrical signal, an intermediate entity, a chemical messenger, an enzyme, a fluorescent entity or “tag,” or a quenching agent. For example, in one embodiment illustrated in FIG. 6A, particle 60 contains one or more quenching agents 62 and one or more nucleic acid recognition entities 61. In this example, quenching agents 62 and nucleic acid recognition entities 61 are bound in such a way that the binding of a nucleic acid to the nucleic acid recognition entity is able to cause one or more quenching agents 62 to be released from particle 60. Of course, the quenching agent and the recognition entity may be bound to other surfaces, such as the surface of a sol or a film. After a binding event to the recognition entity has occurred, quenching agents 62 may be released from the complex at any later time, for example, through a chemical or biological reaction. For example, if an endonuclease such as RNAse H64 is added, cleavage of the double-stranded regions of the complex may occur upon addition of the endonuclease, which may lead to the release of quenching agent 62 from the complex. Other mechanisms of release of quencher are also possible in other embodiments, for example, enzymatic cleavage or spontaneous cleavage of the quenching agent after binding to the recognition entity has occurred.

Once released, the quenching agent may interact with a luminescent polymer or an article containing a luminescent polymer, which may decrease the luminosity of the luminescent polymer. The quenching agent may interact with the luminescent polymer using any mechanism. For example, the quenching agent and the luminescent polymer may be in fluidic or direct communication (i.e., in the same solution, sol, or blend); they may be separated by a membrane; or they may be in different solutions that are later mixed together. If a membrane is used, the membrane may prevent quenching agent from reaching the luminescent polymer unless certain conditions are met, which may be, for example, a function of the binding of the nucleic acid with the recognition entity. For example, the membrane may be a size-exclusion membrane or a molecular weight cut off (MWCO) membrane, a dialysis membrane, a semipermeable membrane, or the like. For example, in FIG. 6B, quenching complex 66 within container 69 (in this particular example, a centrifuge tube) is unable to cross a membrane to reach particles containing luminescent polymer 65. However, upon binding of nucleic acid 65 to the quenching complex 66, quenching agent 62 is released. Quenching agent 62 is able to cross the MWCO membrane, thus leading to a change in luminosity of the particles containing luminescent polymer 65.

As mentioned above, the invention is not limited to only quenching agents. For example, a fluorescent entity may be used instead of a quenching agent, and interaction of the fluorescent entity and the luminescent polymer may cause an increase in the luminosity of the luminescent polymer, e.g., through FRET, and such an increase in luminosity may be determined, e.g., optically.

As another specific example, a recognition entity may be a nucleic acid probe, for instance, a oligomer probe labeled with a quenching agent or a fluorescent entity. The probe may be chimeric, the probe having a unique restriction enzyme site, etc. The probe can be one or more of any combination of DNA, RNA, PNA, or gripNA, including aptamers. The nucleic acid probe may recognize a nucleic acid molecule, for example, viral DNA or RNA, or DNA, rRNA, or mRNA obtained from prokaryotes or eukaryotes, produced by recombinant technology, synthesized chemically, etc. Upon hybridization (binding) of the probe with the nucleic acid molecule, a double-stranded target-probe complex may be formed. In some cases, for example, in an enzyme-based assay, after hybridization, an enzyme may be used to cause the release of the label. The label may then interact with the luminescent polymer, e.g., by increasing or decreasing its luminosity. In some cases, the interaction of the released label with the luminescent polymer may be mechanically or electrophoretically controlled.

As yet another example, an antibody may be used as the recognition entity, e.g., to detect a nucleic acid or a protein. For example, the antibody may be a monoclonal antibody, and the antibody may be labeled, e.g., with a quenching agent or a fluorescent entity. Upon binding of the antibody to the protein or a nucleic acid, the quenching agent or fluorescent entity may be released. As a specific example, in an antibody displacement assay, binding of an analyte to an antibody may cause the release of a pre-bound label, e.g., a pre-bound quenching agent or fluorescent entity, which may then interact with a luminescent polymer, as previously described and be detected.

Any of the systems and methods described herein may be used to determine, measure, sequence, or quantify an analyte such as a protein or a nucleic acid, or a solution suspected of containing a protein or a nucleic acid. In one set of embodiments, the invention may be used for PCR (polymerase chain reaction) analysis or for nucleic acid sequencing methods. For example, the invention may be used to detect or quantify a certain nucleic acid or nucleic acid sequence.

One aspect of the invention contemplates the use of luminescent polymers such as the above-described polymers in articles such as particles, sols, blends, films, or microarrays. The article can be, for example, a solid, a sol, a solution, a suspension, or a dispersion that comprises a luminescent polymer and optionally, a recognition entity such as a nucleic acid recognition entity or a protein recognition entity. If the article is a mixture (for example, as in a dispersion or a blend), then one or more of the components of the mixture may include one or more luminescent polymer types.

In one set of embodiments, the article comprising the luminescent polymer is a particle. A “particle,” as used herein, refers to an isolated, independent structure. The particle may have a diameter of less than 1 mm; in some cases, the diameter may be less than 500 μm; in other cases, less than 50 μm; in other cases, less than 500 nm; and in still other cases, less than 5 nm. In some cases, a particle of the invention may include an aggregate of molecules. For example, in an aggregate, the molecules within the particle may or may not be covalently bound to each other; e.g., the molecules may be aggregated due to covalent bonds, ionic or van der Waals interactions, hydrophobic forces, steric interactions of entangled molecules, and/or some combination of one or more of these. In some embodiments, however, some or all of the molecules defining the particle may be covalently bound to adjacent molecules.

In another set of embodiments, the article is a dispersion, for example, as described in U.S. patent application Ser. No. 09/997,999, entitled “Luminescent Polymer Particles,” by Hancock, et al., filed Nov. 30, 2001. A “dispersion” may comprise one or more particles within a medium, in which the particles and the materials forming the particles (e.g., a polymer) are generally insoluble in the medium, but typically are unable to precipitate out of the medium due to their size and/or other particle/particle interactions that prevent coalescence. The medium containing the particles may be any medium, for example, a fluid such as water or an organic solvent; a gel such as a hydrogel, a polymer such as polystyrene or an optically clear polymer, or a glass such as SiO₂ or other formulations having irregular molecular structures or configurations. In other embodiments, the dispersion may include particles containing embedded polymer molecules unable to significantly react with each other, such as in a silica particle, a latex bead, or the like. In certain cases, the particles may also include additional functionalities. For example, the particle may be coated with another material, or the particle may have a surface chemically altered in some fashion, for example, to provide chemical functional groups suitable for binding additional compounds.

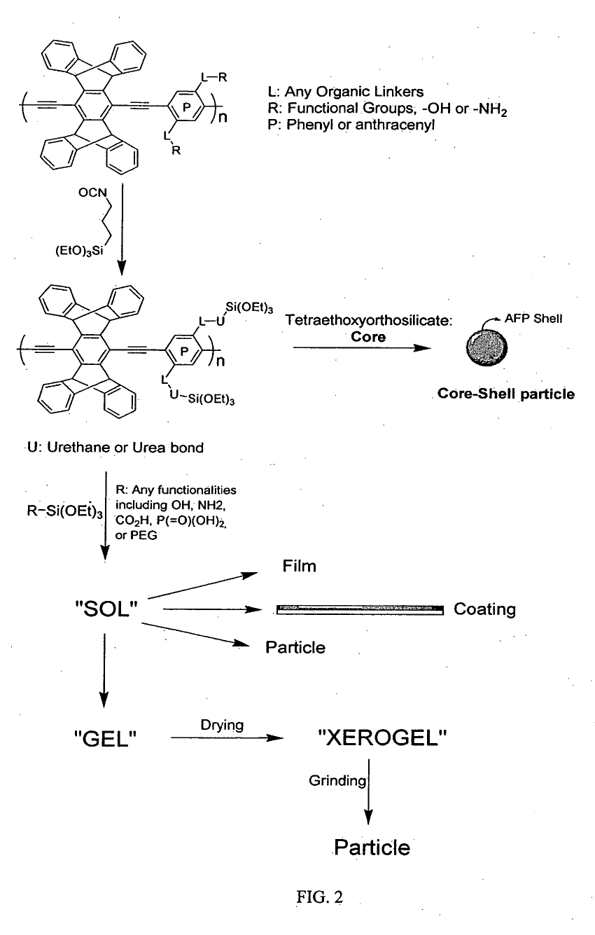

In yet another set of embodiments, the article comprising the luminescent polymer is a sol, for example, a sol particle or film. This can be a silica composite (i.e. a sol, a gel or a xerogel), that may be formed, for example, into a film, a coating, a particle or a core-shell particle. As used herein, “sol” and “gel” are given its ordinary meaning in the field of chemistry. Generally, a sol is comprised of partially cross-linked polysiloxanes and is a free flowing solution, A gel in contrast has reached a crosslink density that forms a non-flowable, semisolid. In one aspect, the silica composite particles include a suspension or dispersion of the particles that are unable to aggregate or precipitate due to their size, charge, or other physical property. In some embodiments, a silica composite can be formed by condensing polymers comprising silanol moieties into a network, such as by reacting the silanol groups into a siloxane network. The silica composite film may be formed by any suitable technique, for example, by spin-casting, or precipitation of a sol. The silica composite particle may be formed by precipitation of a sol, microgel formation or drying of a gel to form a xerogel and then mechanical grinding. For example, in FIG. 2, one method to incorporate a luminescent polymer into a sol is illustrated. A polymer 22, illustrated in FIG. 2 as a poly(phenylene ethynylene) derivative, is reacted with organosilicon compound 24 to produce silanated polymer 26 (in this case, a silanated poly(phenylene ethynylene) derivative). The silicon moieties on the polymer may then be reacted together, for example, using cross-linking agent 28, form a sol network comprising the luminescent polymer.

In still another set of embodiments, the article comprising the luminescent polymer is a blend of at least two polymers. A “blend,” as used herein, is given its ordinary meaning as used in polymer chemistry. A blend typically is a mixture of two or more polymers, where the two or more polymers are not covalently bonded to each other (i.e., as in a copolymer). The polymers within the blend may independently be any phase, for example, a solid, a dispersion, or a sol. In some cases, the two polymers are substantially well-mixed or evenly distributed within the blend (a “homogeneous” blend); in other cases, the two polymers may not be well-mixed or evenly distributed within the blend (e.g., a “heterogeneous” blend).

In one embodiment, the blend includes at least a luminescent polymer and non-luminescent polymer. The luminescent polymer may be any luminescent polymer, for example, one of the luminescent polymers described herein. The non-luminescent polymer may be added to the blend, for example, to improve the physical properties of the polymer, such as to achieve a certain density, viscoelasticity, tensile strength, yield stress, thermal conductivity, or handling characteristic of the blend. The non-luminescent polymer may also be added to improve the optical characteristics of the blend. For example, in one set of embodiments, the non-luminescent polymer is optically transparent or translucent, or confers transparency or translucency on the blend. Any polymer capable of being incorporated along with the luminescent polymer within a blend may be used. For example, the non-luminescent polymer may be a polymer such as, but not limited to, poly(ethylene), poly(ethylene oxide), poly(propylene), poly(propylene oxide), poly(styrene), poly(acrylate), poly(methyl methacrylate), poly(tetrafluoroethylene), poly(vinyl chloride), poly(vinyl fluoride), or the like.

In some cases, the blend may include two or more luminescent polymers. The polymers may be chosen to give the blend certain optical or other physical characteristics, for example, as previously described. For instance, the two polymers may be chosen to result in a blend having certain excitation and/or emission wavelengths, to give the blend the ability to become luminescent at more than one wavelength, and/or to give the blend the ability to emit at more than one wavelength. For example, the excitation and/or emission wavelengths may be chosen so as to be in the optical or visual range (e.g., having a wavelength of between about 400 nm and about 700 nm), infrared range (e.g., having a wavelength of between about 300 μm and 700 nm), ultraviolet range (e.g., having a wavelength of between about 400 nm and about 10 nm), or the like. In some cases, a range of wavelengths may be chosen, for example, between about 350 nm and about 1000 nm, between about 300 μm and about 500 nm, between about 500 nm and about 1 nm, between about 400 nm and about 700 nm, between about 600 nm and about 1000 nm, or between about 500 nm and about 50 nm. In other cases, monochromatic or substantially monochromatic frequencies may be chosen (i.e., having a single wavelength or a narrow wavelength distribution), for example, wavelengths centering around 366 nm, 405 nm, 436 nm, 546 nm, 578 nm, 457 nm, 488 nm, 514 nm, 532 nm, 543 nm, 594 nm, 633 nm, 568 nm, or 647 nm. The monochromatic beam of light may have a narrow distribution of frequencies or wavelengths. For example, 90% or 95% of the wavelengths may be within 5 nm or 3 nm of the average wavelength.

In certain cases, the polymer blend includes two or more similar polymers or copolymers. For example, the polymers may differ in terms of the type or position of the monomers, or in terms of the type or position of side groups within the polymers. In one set of embodiments, the two or more polymers may have distinct excitation and/or emission wavelengths. In another example, one polymer may include an additional monomer unit (e.g., an anthracene subunit), relative to the other polymer. As an example, one polymer may have relatively more anthracene subunits, which may increase the emission wavelength of the polymer.

In one set of embodiments, the article containing the luminescent polymer may be attached to any suitable surface, for example, the surface of a glass slide, a glass capillary, a filter, a polystyrene bead, a cell-culture dish, a polystyrene plate, and the like. In some embodiments, the surface may be activated for the attachment of other molecules, directly or indirectly, for example, through covalent or non-covalent interactions. In certain embodiments, the surface of the solid support may be modified or functionalized with a chemical reagent to provide sites of attachment. For example, the slide may be functionalized with aldehydes, carboxys, epoxys, thiocyanates, isothiocyanates, modified nylons, nitrocelluloses, silanol groups, or the like. In some embodiments, a luminescent polymer may be bound to the microarray, for example, through the use of functional groups on the slide. Of course, in other embodiments, the luminescent polymer may not be bound to the slide or surface.

In one set of embodiments, the article comprising the luminescent polymer is included in an array or a microarray. The terms “microarray” or “array” are given their ordinary meaning as used in the art, and generally refer to an arrangement of entities in a pattern on a substrate. A “microarray” typically is an array where the characteristic length scale is on the order of micrometers. The pattern may be a two-dimensional pattern or a three-dimensional pattern. Any material can be used as a support in the array or microarray, for example, but not limited to, a glass (e.g., a glass slide), a plastic, a polymer, or a metal. In one embodiment, the position of an entity within the array may be used to determine the identity of that entity. The entities may be, for example, discrete drops of fluid or wells located within the substrate. The array or microarray may be used, for example, for sensing or detecting molecules, quantification assays, combinatorial studies, genomics, proteomics, glyconomics, or the like. Molecules that may be attached to a microarray include, for instance, chemical agents, small molecules, biological molecules such as proteins, carbohydrates antibodies, or nucleic acids, or the like. The entities containing the molecules may generally be distinguished from each other in some fashion, for example, compositionally or structurally, or through certain physical characteristics.

In another aspect, the invention comprises a kit. The “kit” typically defines a package including both any one or a combination of the compositions of the invention and instructions of any form that are provided in connection with the composition in a manner such that one of ordinary skill in the art would clearly recognize that the instructions are to be associated with the composition. The instructions can include any oral, written, or electronic communications provided in any manner. The kits described herein may contain one or more containers, which can contain compounds such as the composition as described. The kits also may contain instructions for preparing, mixing, or diluting the compounds. The kits also can include other containers with one or more solvents, surfactants, preservatives, or diluents, as well as containers for mixing or diluting the components to the sample. The compounds in the kit may be provided as liquid solutions or as dried powders. When the compound provided is a dry powder, the powder may be reconstituted by the addition of a suitable solvent, which may or may not be provided. Liquid forms of the compounds may be concentrated or ready to use. The solvent will depend on the compound and the mode of use.

The function and advantage of these and other embodiments of the present invention will be more fully understood from the examples below. The following examples are intended to illustrate the benefits of the present invention, but do not exemplify the full scope of the invention.

### EXAMPLE 1

This example illustrates an embodiment of the invention that uses coupling chemistry. In this example, a probe oligonucleotide was coupled onto a luminescent polymer coating, and hybridized with a complementary target oligonucleotide.

The coupling reaction of a luminescent polymer film with heterobifunctional coupling agents such as maleimidopropionic acid N-hydroxysuccinimide (MPS) produced a thiol-reactive luminescent polymer film (FIG. 9, dotted line). The amine functional group from the polymer film reacted with the succinimidyl moiety of the coupling agent, producing maleimide functional groups on top of the film. The film within the capillary was filled with MPS dissolved dimethyl sulfoxide (DMSO) for 20 min, then washed with fresh DMSO.

The film was then reacted with the probe oligonucleotide in triethanolamine buffer (pH 8.0) overnight at room temperature (25° C.). The probe used in this experiment was a stem-loop 24-mers synthetic oligonucleotide having the following sequence: 5′-FITC-CTTCGTAAGTGGGAAATCTCGAAG-SH-3′ (Seq. ID No. 1). The probe was designed as a stem-loop structure because the probe would be opened and stretched out on the film surface after hybridization. Fluorescence resonance energy transfer (FRET) was used to monitor and evaluate the coupling and hybridization reaction. As shown in FIG. 9 (solid line), a new dominant peak resulted from the energy transfer of the luminescent polymer film to the fluorecein dye, which appeared at 525 nm, while the emission maximum (462 nm) of the luminescent film was found to have decreased. These results indicated directly that photon energy was efficiently transferred from the luminescent polymer to the dye within the stem-loop structure of the probe. In some cases, the energy transfer may diminish once the distance between the polymer and the dye is increased. After hybridization, the probe-tethered polymer film was incubated in Cy5-labeled oligonucleotide solution overnight at room temperature. As shown in FIG. 9 (broken line), the FRET peak decreased after hybridization, while the intensity of the polymer peak recovered.

Thus, this example shows that efficient fluorescence resonance energy transfer may occur from the luminescent polymer to the fluorescence dye of the probe. Additionally, the intensity of the FRET peak decreased after hybridization with the sequence-specific nucleic acid target, illustrating recovery.

### EXAMPLE 2

This example illustrates the photostability of an embodiment of the invention. In this example, Cy3-labeled oligonucleotide and poly(phenylene ethynylene-co-anthracene) were co-spotted in a microarray. The microarray spots were formed by spotting with a DMF solution of polymer D (FIG. 1) and a buffer solution of a Cy-3 labeled oligonucleotide onto aldehyde-functionalized microscope slides. The microarray slide was scanned and exposed to room light for extended periods of time to assess the relative stabilities of the fluorophores, then washed with water. Scans of the slide were taken at 9 hours, 16.5 hours, 22.5 hours, and after washing in water after 22 hours.

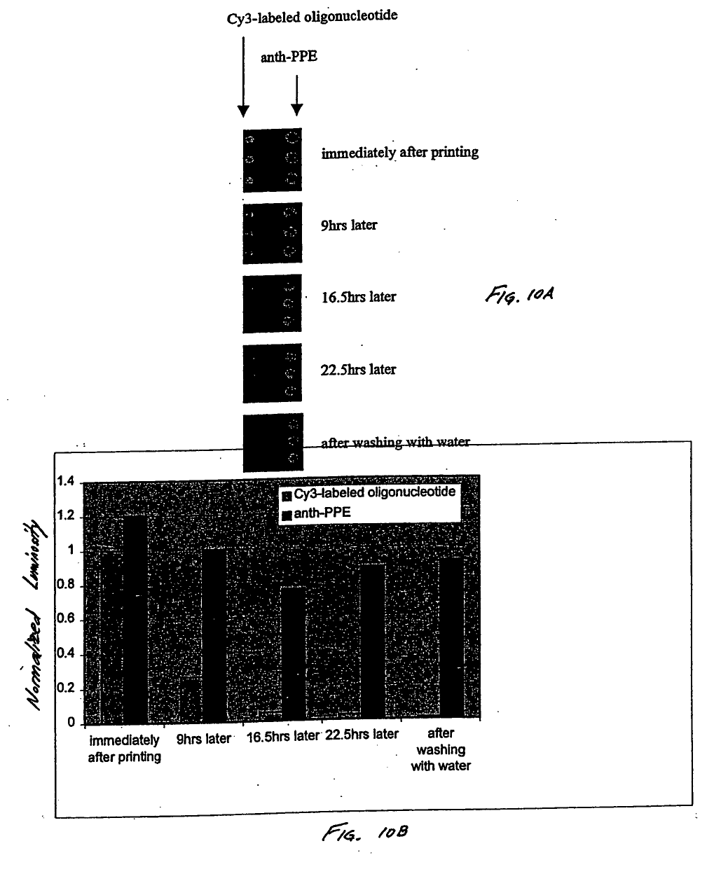

The results of this experiment are illustrated in FIG. 10. FIG. 10A illustrates microarrays containing spots of Cy3-labeled oligonucleotide (spots on the left) and poly(phenylene ethynylene-co-anthracene) (spots on the right) during the experiment. The Cy3-labeled oligonucleotide spots showed a general decrease in luminosity compared to the poly(phenylene ethynylene-co-anthracene) spots. FIG. 10B quantifies this behavior, normalized to Cy3 -labeled oligonucleotide immediately after printing. The Cy3-labeled oligonucleotide spots showed a decrease in luminosity to less than 5% after 22.5 hours and washing in water. In contrast, the poly(phenylene ethynylene-co-anthracene) spots did not show a statistically significant decrease in relative luminosity.

These experiments demonstrate the compatibility of the inventive materials with microarray spotters and scanners, such as those commonly used in the laboratory. Polymer D in FIG. 10B was found to exhibit substantially more stable fluorescence under these experimental conditions when compared to the Cy-3 labeled oligonucleotide. In addition, polymer D did not wash off the surface of the slide upon rinsing with water.

Thus, these results demonstrate that the inventive materials described herein can be spotted using a common laboratory array spotter. The utility of using the Cy-3 channel of a common commercial microarray scanner to visualize the resulting spots was also demonstrated in this example. Additionally, these results demonstrate that spots containing fluorophores can be exposed to room or ambient light for extended periods of time without significant photobleaching and/or photodegredation. Finally, as demonstrated here, the spotted slides can be washed with water without detectable loss of the immobilized polymer.

### EXAMPLE 3

This example illustrates binding and sensitivity of a labeled oligonucleotide to a luminescent polymer particle of the invention.

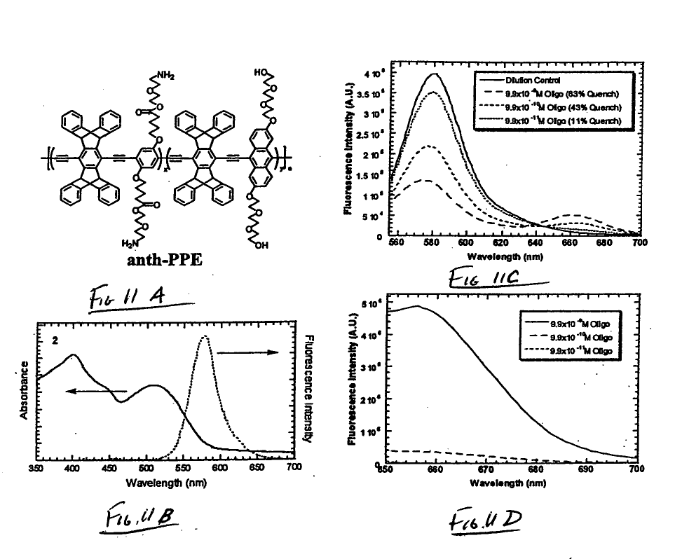

FIG. 11A illustrates the molecular structure of poly(phenylene ethynylene-co-anthracene) used in this experiment. FIG. 11B illustrates the absorbance and emission spectra of this polymer as dispersed particles in water. FIGS. 11C and 11D show titration of the aqueous dispersion using a Cy5-labeled oligonucleotide. FIG. 11C shows quenching of the emission of the polymer, while FIG. 11D illustrates the direct excitation of the Cy5 fluorophore. The detection limit from the fluorescence quenching measurements was observed to be two orders of magnitude lower.

### EXAMPLE 4

This example illustrates the preparation and use of a nucleic acid probe of the invention.

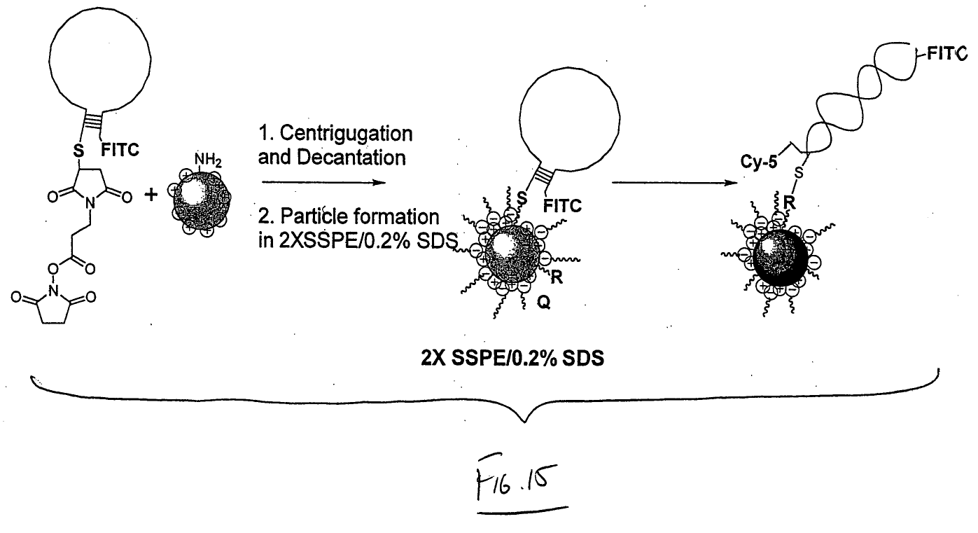

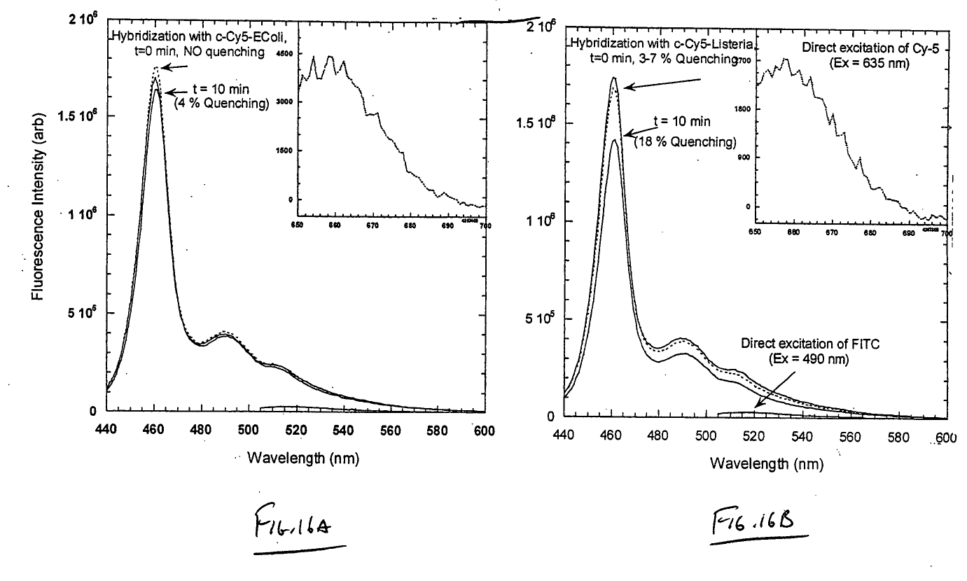

A nucleic acid probe was prepared by binding luminescent polymer-sol particles to a nucleic acid sequence having a stem-loop configuration (FIG. 15). The nucleic acid sequence was selected to bind a target nucleic acid sequence from Listeria. The probe-attached luminescent polymer-sol particles dispersed in 2XSSPE/0.2% SDS buffer were hybridized with a Cy5-labeled nontarget sequence (E. Coli sequence) and a Cy5-labeled true target sequence, respectively, at room temperature. The true Cy5-labeled target caused fluorescence quenching of 18% upon hybridization (FIG. 16B), whereas the control did not show any significant changes (FIG. 16A), thus indicating the specificity of the inventive probe to the target nucleic acid sequence.

### EXAMPLE 5

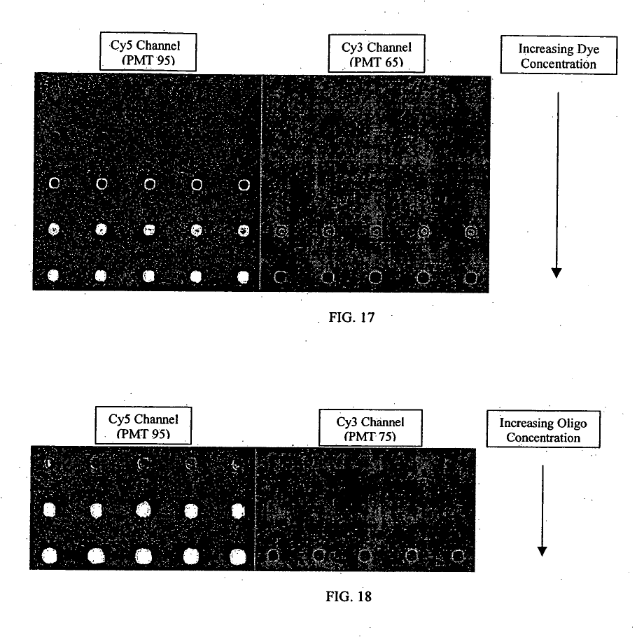

This example illustrates the versatility of fluorescence quenching-based signal transduction as it pertains to microarray-based experiments for gene expression, toxicogenomics, etc. Numerous fluorescent—and non-fluorescent—dyes have exhibited efficient quenching of the polymer fluorescence signature including Cy-dyes, BHQ-dyes, QSY-dyes and xylene cyanole. In the example shown in FIG. 17, various concentrations of a fluorescent dye (xylene cyanole) were spotted onto a microscope slide coated with a polymer-sol-gel mixture (with or without a poly-lysine top layer) and scanned using a commercial microarray reader. The dye is visible in the Cy5 channel of the scanner, while the polymer-coating is visible in the Cy3 channel. For example, dye (spotting) concentrations of 5×10⁻⁷M and 5×10⁻⁶M result in 31% and 76% polymer fluorescence quenches respectively. This experiment demonstrates flexibility over the choice of quencher dye, enabling the use of alternative stable, non-proprietary dyes.

### EXAMPLE 6

This example (FIG. 18) illustrates fluorescence quenching of a polymer-coated slide via adsorption of a labeled nucleic acid. Various concentrations of a Cy5-labeled nucleic acid sequence were spotted onto a polymer-sol-gel coated microscope slide and scanned using a commercial microarray reader. Once again, the dye is visible in the Cy5 channel of the scanner, while the polymer-coating is visible in the Cy3 channel. For example, nucleic acid (spotting) concentrations as low as 5×10⁻⁸M result in measurable quenching of the polymer-coating's fluorescence. See Table 1 below. This experiment demonstrates that labeled nucleic acid—adsorbed onto a polymer-sol-gel coated microscope slide—results in efficient signal transduction (quenching).

TABLE 1

| Nucleic Acid | Fluorescence Quench |
|---|---|
| Concentration (M) | (%) |
| 5 × 10⁻⁸ | 7.5 |
| 5 × 10⁻⁷ | 41 |
| 5 × 10⁻⁶ | 74 |

### EXAMPLE 7

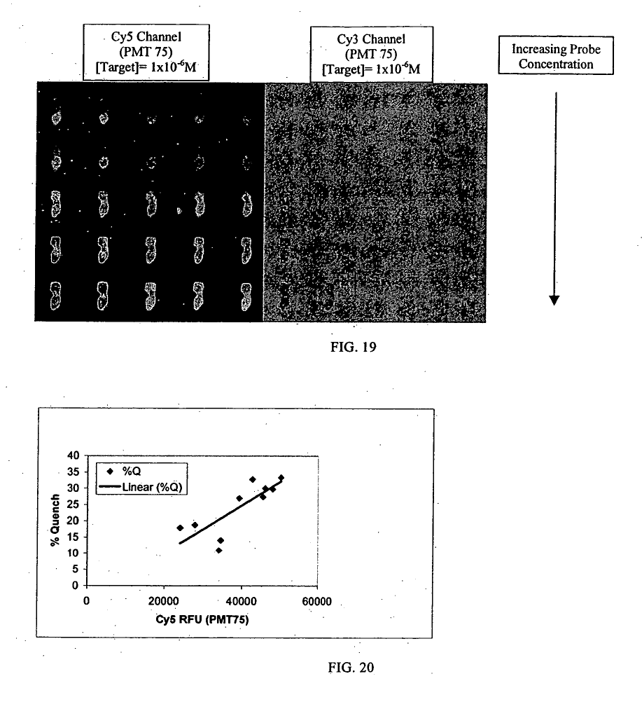

This example illustrates signal transduction via a hybridization event. A base-treated microscope slide was immersed for 5 minutes in a DMSO solution of polymer-sol-gel material, rinsed with DMSO, spun-dry and oven annealed for 30 minutes. The resulting polymer-coated slide was activated towards covalent coupling by immersion in a 1 g/5 mL 1-methyl 2-pyrolidinone solution of cyanuric chloride for 1 hour, rinsed with 1-methyl 2-pyrolidinone and spun-dry. In other examples, poly-lysine-based photocrosslinking was utilized for probe deposition. Various concentrations—ranging from 2×10⁻⁸M to 2×10⁻⁴M—of amino-functionalized probe sequence were spotted onto this activated surface and flash heated in the oven for 5 minutes. Next, the spotted region was covered with a hybridization chamber and the surface blocked by the addition of a 1× hybridization solution of salmon testes DNA (0.99 mg/mL) for 30 minutes with subsequent rinsing with 1×hybridization solution. Finally, various concentrations—ranging from 1×10⁻⁸M to 1×10⁻⁶M—of Cy5-labeled target (or non-target) nucleic acid sequence were introduced, incubated at 42° C. for 14.5 hours, rinsed with 1×SSPE/0.2% SDS then 0.1×SSPE/0.2% SDS, spun-dry and scanned in a commercial microarray scanner. Images obtained via the introduction of Cy5-labeled true target (1×10⁻⁶M) are shown in FIG. 19. When the results obtained from direct excitation of the dye (coupled to target) were compared with the resultant polymer-coating's fluorescence quench, they correlated exactly. See FIG. 20. This example illustrates that we can prepare polymer-coated slides, attach probe nucleic acid sequences to them, block and finally hybridize to dye-labeled complementary nucleic acid sequence, resulting in signal transduction (quenching). This illustrates the utility of the system in microarray-based hybridization experiments for applications such as gene expression analysis and toxicogenomics.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, section 2111.03.

What is claimed is:

1. an article, comprising:
a composition comprising a luminescent polymer and a recognition entity, wherein the composition is a sol, a blend, or a film.

2. The article of claim 1, wherein the recognition entity comprises a nucleic acid recognition entity.

3. The article of claim 1, wherein the luminescent polymer comprises an iptycene moiety.

4-6. (canceled)

7. The article of claim 1, wherein the luminescent polymer comprises a backbone.

8. The article of claim 7, wherein the backbone comprises a delocalized pi-electron bond.

9. The article of claim 7, wherein the backbone comprises a benzene ring.

10. The article of claim 7, wherein the backbone comprises a triple bond.

11. (canceled)

12. (canceled)

13. The article of claim 1, wherein the luminescent polymer comprises a structure: 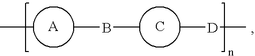
wherein n is at least 1, at least one of A and C comprises a bicyclic ring system, and at least one of B and D comprises a triple bond

14. (canceled)

15. The article of claim 13, wherein at least two of A, B, C, and D are in pi-electron communication.

16-19. (canceled)

20. The article of claim 2, wherein the nucleic acid recognition entity comprises a nucleic acid.

21. The article of claim 2, wherein the nucleic acid recognition entity comprises a deoxyribonucleic acid.

22. The article of claim 2, wherein the nucleic acid recognition entity comprises a ribonucleic acid.

23-27. (canceled)

28. The article of claim 2, wherein the nucleic acid recognition entity recognizes a nucleic acid sequence of at least about 10 bases.

29. (canceled)

30. (canceled)

31. The article of claim 2, wherein the nucleic acid recognition entity is charged.

32. (canceled)

33. (canceled)

34. The article of claim 2, wherein the nucleic acid recognition entity is able to change conformation upon binding of a nucleic acid to a nucleic acid recognition entity.

35. (canceled)

36. (canceled)

37. An article, comprising:
a sol comprising a luminescent polymer.

38-45. (canceled)

46. An article, comprising:
a luminescent polymer;
a recognition entity selected from the group consisting of a nucleic acid recognition entity, a protein recognition entity, and an aptamer; and
an energy migration pathway between the luminescent polymer and the recognition entity.

47-67. (canceled)

68. An article, comprising:
a substrate having a surface charge, the substrate comprising a luminescent polymer.

69-84. (canceled)

85. A method, comprising:
providing a article comprising a luminescent polymer and a nucleic acid recognition entity, the article having a luminosity;
allowing a nucleic acid molecule to bind to the nucleic acid recognition entity; and
detecting a change in luminosity of the article.

86-100. (canceled)

101. A method, comprising:
providing a article comprising a luminescent polymer and a recognition entity, the article having a luminosity;
allowing an analyte to bind to the recognition entity; and
detecting a change in luminosity of the article.


SEQUENCE LISTING (1 sequence)

SEQ ID NO: 1 - DNA, 24 bp, Artificial Sequence
  Synthetic Oligonucleotide
cttcgtaagtgggaaatctcgaag